(19) United States
(12) Patent Application Publication
(10) Pub. No.: US 2012/0309790 A1
(43) Pub. Date: Dec. 6, 2012

(54) TREATMENT OF DERMATOLOGICAL ALLERGIC CONDITIONS

(75) Inventors: Yong Mi CHOI-SLEDESKI, Belle Mead, NJ (US); Neil FITCH, Bridgewater, NJ (US); Anne MINNICH, Flemington, NJ (US)

(73) Assignee: SANOFI, Paris (FR)

(21) Appl. No.: 13/589,525

(22) Filed: Aug. 20, 2012

Related U.S. Application Data
(63) Continuation of application No. PCT/US2011/025785, filed on Feb. 23, 2011.
(60) Provisional application No. 61/307,650, filed on Feb. 24, 2010.

(30) Foreign Application Priority Data
Dec. 2, 2010 (FR) 1060021

(51) Int. Cl. A61K 31/454; A61P 17/06; A61P 37/08; A61P 17/00

(52) U.S. Cl. 514/323

(57) ABSTRACT

A treatment for a dermatological allergic condition is disclosed, using a compound of Formula I:

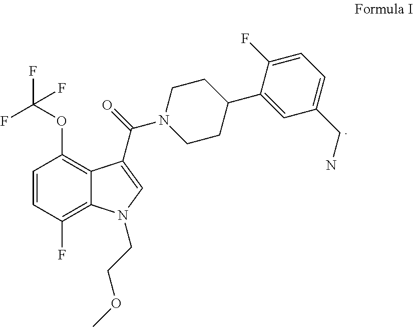

### FIELD OF THE INVENTION

This invention is directed to a method of therapy for human and non-human patients suffering from, or subject to, dermatological allergic conditions.

### BACKGROUND OF THE INVENTION

Mast cell mediated inflammatory conditions are a growing public health concern. Tryptase is stored in mast cell secretory granules and is the major protease of human mast cells. Tryptase has been implicated in a variety of biological processes, including degradation of vasodilatory and bronchodilatory neuropeptides (Caughey, et al., J. Pharmacol. Exp. Ther., 1988, 244, pages 133-137; Franconi, et al., J. Pharmacol. Exp. Ther., 1988, 248, pages 947-951; and Tam, et al., Am. J. Respir. Cell Mol. Biol., 1990, 3, pages 27-32).

As a result, tryptase inhibitors may be useful as anti-inflammatory agents (K Rice, P. A. Sprengler, Current Opinion in Drug Discovery and Development, 1999, 2(5), pages 463-474), and may be useful in treating or preventing dermatological allergic conditions such as atopic dermatitis (A. Jarvikallio et al, Br. J. Dermatol., 1997, 136, pages 871-877).

Such a compound should readily have utility in treating a patient suffering from conditions that can be ameliorated by the administration of an inhibitor of tryptase, e.g., mast cell mediated inflammatory conditions, inflammation, and diseases or disorders related to the degradation of vasodilatory neuropeptides, and have diminished liability for semicarbazide-sensitive amine oxidase (SSAO) metabolism.

The compound of formula I (Compound A) is a selective and reversible inhibitor of human beta-tryptase and mouse MCPT-6 (mouse ortholog of human beta-tryptase) with Ki on recombinant enzymes of 38 and 920 nM, respectively.

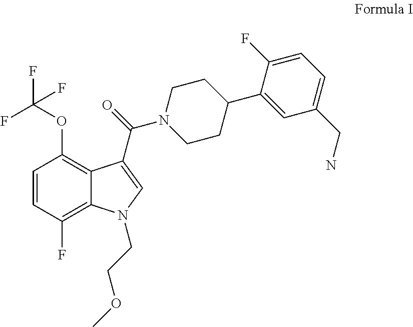

### SUMMARY OF THE INVENTION

We have now found that a compound of Formula I, or its pharmaceutically acceptable salts, is useful for the treatment of inflammatory bowel disease.

Namely, this invention relates to a prophylactic or therapeutic drug for dermatological allergic conditions, in particular atopic dermatitis, containing, as the active constituent, a compound or salt thereof represented by Formula I.

A method is also disclosed for the treatment of dermatological allergic conditions, in particular atopic dermatitis, in a mammal comprising the step of administering a pharmaceutically effective amount of a compound represented by Formula I below or as pharmaceutically acceptable salt thereof.

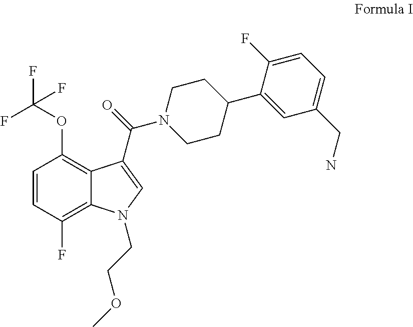

This compound is also known as [4-(5-Aminomethyl-2-fluorophenyl)piperidine-1-yl][7-fluoro-1-(2-methoxyethyl)-4-trifluoromethoxy-1H-indol-3-yl]methanone.

This invention is directed to a compound of Formula I, which has now been found to be active in an animal model for dermatological allergic conditions, and in particular for atopic dermatitis.

Another aspect of the present invention is a pharmaceutical composition for treating dermatological allergic conditions.

Another aspect of the present invention is a treatment for atopic dermatitis.

Yet another aspect of the present invention is a treatment for atopic dermatitis by treating a patient with a beta-tryptase inhibitor in general.

### DETAILED DESCRIPTION OF THE INVENTION

Thus, in one aspect, the present invention is directed to pharmaceutical compositions comprising a compound of general Formula I, which also may be known as: [4-[5-Aminomethyl-2-fluorophenyl)piperidine-1-yl][7-fluoro-1-(2-methoxyethyl)-4-trifluoromethoxy-1H-indol-3-yl]methanone or Compound A.

In the present specification, the term “compound of the invention”, and equivalent expressions, are meant to embrace a compound of general formula (I) as hereinbefore described, which expression includes the ester prodrugs, the pharmaceutically acceptable salts, and the solvates, e.g. hydrates, where the context so permits. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

### Preparatory Details

The compound of formula I may be prepared by the application or adaptation of known methods, by which is meant methods used heretofore or described in the literature, for example those described by R. C. Larock in Comprehensive Organic Transformations, VCH publishers, 1989, or as described herein.

In the reactions described hereinafter it may be necessary to protect reactive functional groups, for example, amino groups, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991.

In particular, the compound of formula I may be prepared as shown through Schemes 1-2.

For example, the compound of the present invention is an achiral compound whose preparation is comprised of a convergent synthesis. The compound of the invention, as its benzoate salt, is prepared as shown in the schemes below.

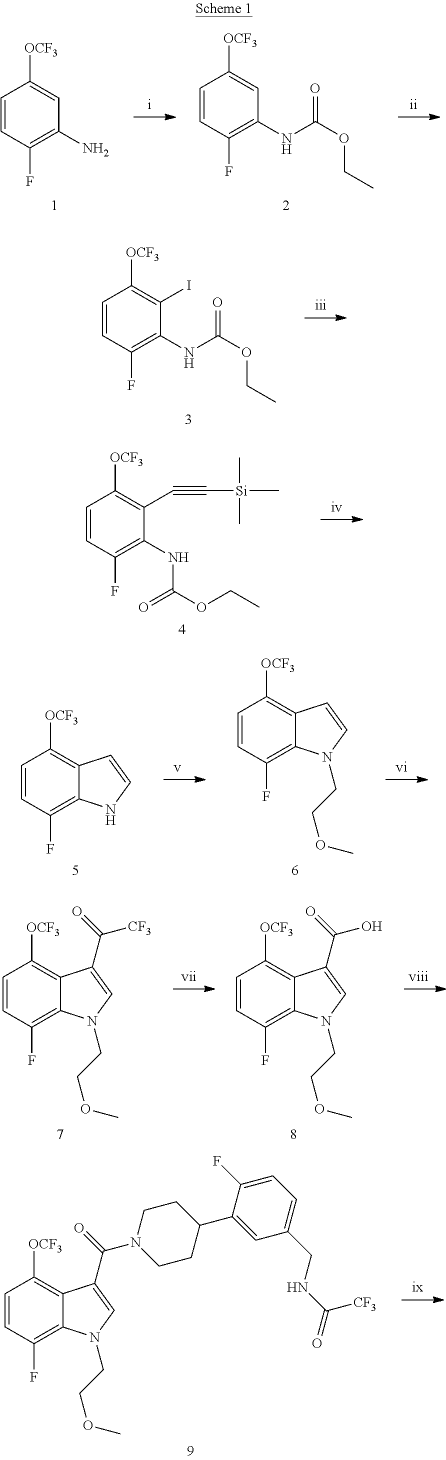

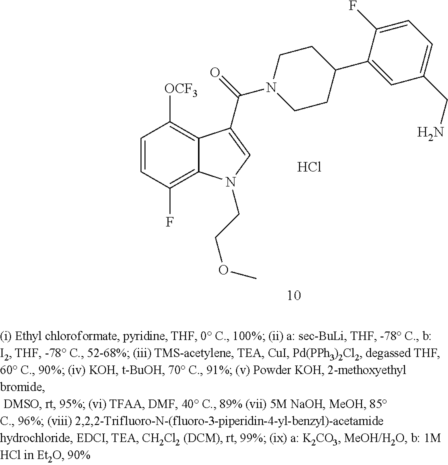

Compound 1 is converted to compound 2 by protecting the amino group with an amino protecting agent, such as ethyl chloroformate in the presence of a suitable base, such as pyridine, to yield protected compound 2.

Compound 2 is converted to compound 5 in a three step process. Compound 2 is iodinated in the position next to the carbamic ester by reacting 2 with a strong base such as secondary butyl lithium to form the anion which is reacted with an iodide source such as molecular iodine to give compound 3. Compound 3 is then converted to acetylenic compound 4 using catalytic conditions such as copper (I) iodide and bistriphenylphosphine palladium (II) dichloride in the presence of trimethylsilylacetylene and base such as triethylamine. Compound 4 is cyclized using a strong base such as potassium hydroxide and heating to give indole compound 5.

Compound 5 is converted to compound 6 by alkylating the indole nitrogen thereof with an alkyl halide in the presence of a strong base, such as a potassium hydroxide, in a dipolar aprotic solvent, such as dimethylsulfoxide, at room temperature to yield compound 6.

Compound 6 is converted to compound 8 in a two step process. First, compound 6 is converted to compound 7 by treating compound 6 with trifluoroacetic anhydride in the presence of a solvent such as N,N-dimethylformamide and heating. Compound 7 is treated with a strong base such as sodium hydroxide to give compound 8 which has an acid function in the 3-position thereof.

Compound 8 is converted to amide 9 by reacting acid 8 with 2,2,2-trifluoro-N-(fluoro-3-piperidin-4-yl-benzyl)-acetamide hydrochloride (compound 14) in the presence of an acid coupling reagent such as EDCI and an organic base such as triethylamine in an inert solvent such as dichloromethane.

Compound 9 is converted to compound 10 by deprotecting N-benzyl trifluoroacetamide on treatment with mild base, such as potassium carbonate, in solvent mixture, such as methanol/water. The hydrochloride salt can be formed in the presence of a polar organic solvent, such as ether, to yield compound 10 which is the hydrochloride salt of ([4-(5-aminomethyl-2-fluoro-phenyl)-piperidin-1-yl]-[7-fluoro-1-(2-methoxy-ethyl)-4-methyl-1H-indol-3-yl]-methanone) in formula I.

The reactions of this scheme are as follows.

### Step A: Preparation of (2-Fluoro-5-trifluoromethoxy-phenyl)-carbamic acid ethyl ester (2)

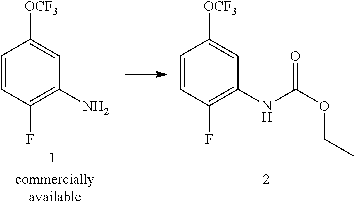

To a solution of 1 (50.72 g, 0.26 mol) and pyridine (27.3 mL, 0.34 mol) in THF (500 mL) at 0° C. is added ethyl chloroformate (32.2 mL, 0.39 mol) dropwise over a 30 min period. After 1 h, both LC/MS and TLC indicate that the reaction is completed. The reaction mixture is partitioned between H₂O and EtOAc. The two layers are separated, and the organic layer is washed with 1 M HCl, H₂O, and brine, dried over MgSO₄, filtered, and concentrated in vacuo. The crude material is purified on silica gel with heptane/EtOAc (95/5 to 70/30) as eluant to give 69.23 g (99%) of the product 2 as a clear colorless liquid. ¹H NMR (CDCl₃) δ 8.11 (br s, 1H), 7.07 (dd, J=9.1, 9.3 Hz, 1H), 7.00-6.80 (m, 2H), 4.27 (q, J=7.1 Hz, 2H), 1.33 (t, J=7.1 Hz, 3H); ¹⁹F NMR (CDCl₃) δ −57.84 (s, 3F), −134.01 (br s, 1F); MS 309 (M+CH3CN+1, 100%), 268 (M+1).

### Step B: Preparation of (6-Fluoro-2-iodo-3-trifluoromethoxy-phenyl)-carbamic acid ethyl ester (3)

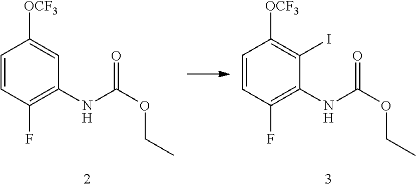

To a solution of 2 (31.34 g, 117.2 mmol) in THF (180 mL) at −78° C. is added sec-BuLi (1.4 M in cyclohexane, 200 mL, 280 mmol) dropwise over a 1 h period. After 20 min, a solution of I₂ (44.6 g, 175.8 mmol) in THF (150 mL) is added dropwise over a 30 min period. This mixture is then stirred at −78° C. for 30 min. Saturated NH₄Cl is added, and the cooling bath is removed. The reaction mixture is partitioned between H₂O and EtOAc. The two layers are separated, and the organic layer is washed with 10% Na₂SO₃, H₂O, and brine, dried over MgSO₄, filtered, and concentrated in vacuo. The residue is suspended in DCM (50 mL), and heptane (300 mL) is added. The white powder 3 (18.1 g, 39%) from the resulting suspension is collected by suction filtration and air-dried. The filtrate is concentrated in vacuo, and the residue is suspended in heptane (200 mL). Another batch of 3 (3.8 g, 8%) is collected by suction filtration and air-dried. Additional product can be obtained by purifying the filtrate via silica gel chromatography. ¹H NMR (CDCl₃) δ 7.30-17.10 (m, 2H), 6.16 (br s, 1H), 4.26 (q, J=7.1 Hz, 2H), 1.32 (t, J=7.1 Hz, 3H); ¹⁹F NMR (CDCl₃) δ −56.90 (s, 3F), −114.35 (d, J=8.5 Hz, 1F); MS 394 (M+1, 100%), 374, 364, 321, 267.

### Step C: Preparation of (6-Fluoro-3-trifluoromethoxy-2-trimethylsilanylethynyl-phenyl)-carbamic acid ethyl ester (4)

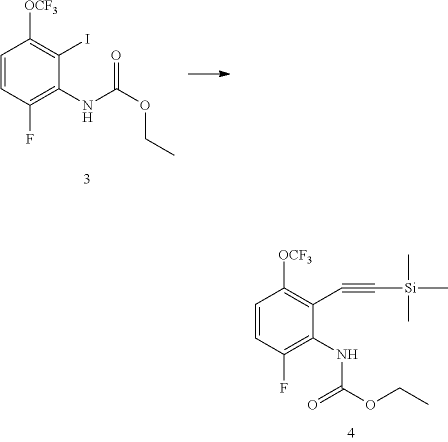

A mixture of 3 (18.1 g, 45.9 mmol), Et₃N (12.8 mL, 91.9 mmol), Pd(PPh)₂Cl₂ (1.6 g, 5% mol), CuI (0.7 g, 8% mol), and TMS-acetylene (19.6 mL, 137.8 mmol) in degassed THF (180 mL) is heated at 60° C. overnight. The mixture is cooled to rt, and then partitioned between H₂O and EtOAc. This mixture is filtered through Celite to remove the insoluble material. The two layers of the filtrate are separated, and the organic layer is washed H₂O and brine, dried over MgSO₄, filtered, and concentrated in vacuo. The crude material is purified on silica gel with heptane/EtOAc as eluant to give 15.6 g (93%) of the product 4 as beige solid. ¹H NMR (CDCl₃) δ 7.15-7.00 (m, 2H), 6.41 (br s, 1H), 4.26 (q, J=7.1 Hz, 2H), 1.31 (t, J=7.1 Hz, 3H), 0.27 (s, 9H); ¹⁹F NMR (CDCl₃) δ −57.59 (s, 3F), -118.15 (s, 1F); MS 364 (M+1, 100%).

### Step D: Preparation of 7-Fluoro-4-trifluoromethoxy-1H-indole (5)

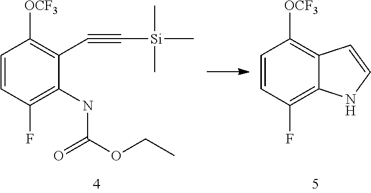

A mixture of 4 (28.9 g, 79.6 mmol) and KOH (35.7 g, 636.7 mmol) in degassed t-BuOH (300 mL) is heated at 70° C. overnight. LC/MS indicates the reaction is completed. The mixture is cooled to rt, and then partitioned between H₂O and Et₂O. The two layers are separated, and the aqueous layer was extracted with Et₂O (2×). The combined organic layers are washed with H₂O and brine, dried over MgSO₄, filtered, and concentrated in vacuo. The crude material is purified on silica gel with heptane/EtOAc (100/0 to 60/40) as eluant to give 16 g (91%) of 5 as a yellow liquid. ¹H NMR (CDCl₃) δ 8.47 (br s, 1H), 7.35-7.20 (m, 1H), 6.95-6.80 (m, 2H), 6.68 (d, J=2.5 Hz, 1H); ¹⁹F NMR (CDCl3) δ −57.63 (s, 3F), -136.10 (d, J=8.5 Hz, 1F); MS 220 (M+1, 100%), 200.

### Step E: Preparation of 7-Fluoro-1-(2-methoxy-ethyl)-4-trifluoromethoxy-1H-indole (6)

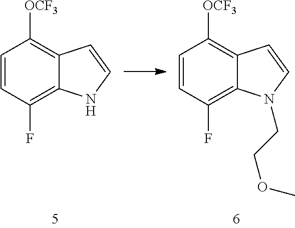

A mixture of 5 (16 g, 72.8 mmol) and powder KOH (20.4 g, 364.2 mmol) in DMSO (150 mL) is stirred at rt for 10 min. 2-Methoxyethyl bromide (10.3 mL, 109.2 mmol) is added. This mixture is stirred at rt overnight. LC/MS indicates the reaction is completed. The mixture is partitioned between H₂O and Et₂O. The two layers are separated, and the aqueous layer is extracted with Et₂O (2×). The combined organic layers are washed with H₂O and brine, dried over MgSO₄, filtered, and concentrated in vacuo. The crude material is purified on silica gel with heptane/EtOAc (100/0 to 50/50) as eluant to give 19.3 g (95%) of 6 as a yellow liquid. ¹H NMR (CDCl₃) δ 7.15 (d, J=2.1 Hz, 1H), 6.90-6.75 (m, 2H), 6.56 (t, J=2.5 Hz, 1H), 3.72 (t, J=5.2 Hz, 2H), 3.72 (t, J=5.2 Hz, 2H), 3.31 (s, 3H); ¹⁹F NMR (CDCl₃) δ −57.54 (s, 3F), −137.00 (d, J=11.3 Hz, 1F); MS 278 (M+1, 100%).

### Step F: Preparation of 2,2,2-Trifluoro-1-[7-fluoro-1-(2-methoxy-ethyl)-4-trifluoromethoxy-1H-indol-3-yl]ethanone (7)

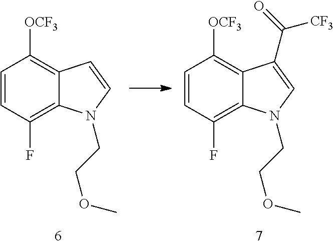

To a mixture of 6 (19.3 g, 69.7 mmol) in DMF (135 mL) is added TFAA (26.2 mL, 188.2 mmol). This mixture is heated at 40° C. overnight. TLC indicates the reaction is completed. The mixture is cooled to rt, and then partitioned between H₂O and Et₂O. The two layers are separated, and the organic layer is washed with saturated NaHCO₃ (2×), H₂O and brine, dried over MgSO₄, filtered, and concentrated in vacuo. The crude material is purified on silica gel with heptane/EtOAc (100/0 to 50/50) as eluant to give 23.4 g (89%) of 7 as a slightly green solid. ¹H NMR (CDCl₃) δ 8.03 (d, J=1.4 Hz, 1H), 7.20-6.95 (m, 2H), 4.54 (t, J=4.9 Hz, 2H), 3.76 (t, J=4.8 Hz, 2H), 3.33 (s, 3H); ¹⁹F NMR (CDCl₃) δ −57.74 (s, 3F), −71.10 (s, 3F), −134.95 (d, J=11.5 Hz, 1F); MS 374 (M+1, 100%).

### Step G: Preparation of 7-Fluoro-1-(2-methoxy-ethyl)-4-trifluoromethoxy-1H-indole-3-carboxylic acid (8)

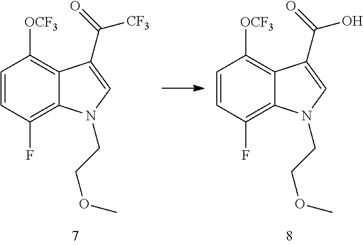

A mixture of 7 (23.4 g, 62.6 mmol) in MeOH (100 mL) and 5 M NaOH (100 mL) is heated at 80° C. overnight. LC/MS indicates that the reaction is complete. The reaction mixture is cooled to rt, and then concentrated in vacuo to remove most of the MeOH. The residue is dissolved in H₂O, and then washed with Et₂O once. The aqueous layer is slowly acidified to pH ˜2 with conc. HCl. The acidified suspension is extracted with Et₂O, and the organic extract is washed with H₂O and brine, dried over MgSO₄, filtered, and concentrated in vacuo. The residue is suspended in DCM/heptane (10/90). The white powder 8 (19.4 g, 96%) in the suspension is collected by suction filtration and air-dried. ¹H NMR (CDCl₃) δ 8.02 (s, 1H), 7.15-7.05 (m, 1H), 7.00-6.90 (m, 1H), 4.49 (t, J=5.0 Hz, 2H), 3.75 (t, J=4.9 Hz, 2H), 3.33 (s, 3H); ¹⁹F NMR (CDCl₃) δ −57.74 (s, 3F), -135.65 (d, J=11.3 Hz, 1F); MS 363 (M+CH₃CN+1), 322 (M+1, 100%).

### Step H: Preparation of 2,2,2-Trifluoro-N-(4-fluoro-3-{1-[7-fluoro-1-(2-methoxy-ethyl)-4-trifluoromethoxy-1H-indole-3-carbonyl]-piperidin-4-yl}-benzyl)-acetamide (9)

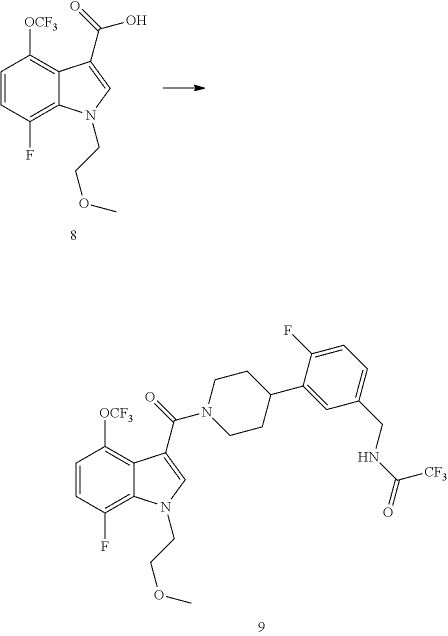

A mixture of 8 (19.1 g, 59.6 mmol), Et₃N (24.8 mL, 177.9 mmol), 2,2,2-trifluoro-N-(4-fluoro-3-piperidin-4-yl-benzyl)-acetamide hydrochloride (11, 26.4 g, 77.5 mmol) (14), and EDCI (17.1 g, 89.3 mmol) in CH₂Cl₂ is stirred at rt overnight. Both TLC and LC/MS indicate that the reaction is completed. The mixture is partitioned between H₂O and CH₂Cl₂. The two layers are separated, and the organic layer is washed with brine, dried over MgSO₄, filtered, and concentrated in vacuo. The crude material is purified on silica gel with heptane/EtOAc (40/60 to 0/100) as eluant to give 9 (36 g, 99%) as a white foam. ¹H NMR (CDCl₃) δ 7.37 (s, 1H), 7.20-7.10 (m, 2H), 7.10-6.85 (m, 4H), 4.95 (br s, 1H), 4.60-4.35 (m, 4H), 3.90 (br s, 1 H), 3.73 (t, J=5.0 Hz, 2H), 3.32 (s, 3H), 3.25-2.70 (m, 3H), 2.05-1.50(m, 4H); ¹⁹F NMR (CDCl₃) δ −57.54 (s, 3F), −75.39 (s, 3F), −119.31 (s, 1F), −134.96 (d, J=11.3 Hz, 1F); MS 608 (M+1, 100%).

### Step I: Preparation of [4-(5-Aminomethyl-2-fluoro-phenyl)-piperidin-1-yl]-[7-fluoro-1-(2-methoxy-ethyl)-4-trifluoromethoxy-1H-indol-3-yl]-methanone hydrochloride salt (10)

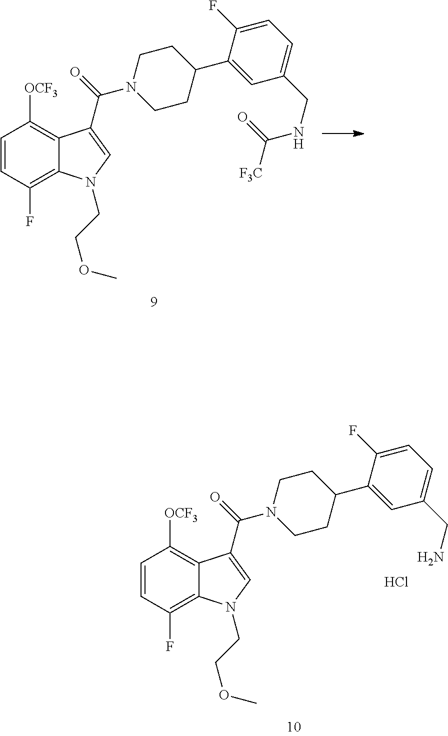

To a mixture of 9 (36 g, 59.3 mmol) in MeOH (400 mL) is added aqueous K₂CO₃ (65.5 g, 474 mmol, dissolved in 120 mL H₂O). This mixture is stirred at rt overnight. LC/MS indicates the reaction is completed. The reaction mixture is concentrated in vacuo to remove most of the methanol. The residue is partitioned between H₂O and EtOAc. The two layers are separated, and the organic layer is washed with H₂O and brine, dried over MgSO₄, filtered, and concentrated in vacuo to yield 27.5 g (90%) of 10 as a clear colorless sticky gum. ¹H NMR (CDCl₃) δ 7.42 (s, 1H), 7.25-7.10 (m, 2H), 7.05-6.85 (m, 3H), 4.92 (br s, 1H), 4.46 (t, J=5.2 Hz, 2H), 3.86 (br s, 3H), 3.74 (t, J=5.1 Hz, 2H), 3.32 (s, 3H), 3.30-2.75 (m, 3H), 2.24 (br s, 2H), 2.05-1.55 (m, 4H); ¹⁹F NMR (CDCl₃) δ −57.52 (s, 3F), −121.64 (s, 1F), −136.03 (d, J=11.3 Hz, 1F); MS 512 (M+1, 100%).

To a solution of the above material (2.856 g, 5.59 mmol) in Et₂O (30 mL) is added 2 N HCl/Et₂O (3 mL, 6 mmol) dropwise. A solid precipitate forms and the ethereal solution is decanted off. The solid is washed with additional Et₂O then decanted off. The remaining pale yellow solid is dissolved in warm MeOH (10 mL) then Et₂O (50 mL) is added until the solution is slightly cloudy. After ca. 2 hrs solid precipitate appears. Additional Et₂O (5-10 mL) is added and then the suspension is placed in the fridge overnight. A white crystalline product (2.475 g, 4.52 mmol) is collected and dried under high vacuum for 4 hrs. ¹H NMR (DMSO-d₆) δ 8.32 (br s, 2H), 7.71 (s, 1H), 7.43 (d, 1H, J=7.2 Hz), 7.36 (m, 1H), 7.26-7.20 (m, 1H), 7.12-7.08 (m, 2H), 4.49 (t, J=5.1Hz, 2H), 4.00 (s, 2H), 3.71 (t, J=5.1Hz, 2H), 3.32 (s, 3H), 3.21-3.07 (m, 3H), 2.99 (br s, 2H), 1.80-1.62 (m, 4H); ¹⁹F NMR (DMSO-d₆) δ −56.79 (s, 3F), −119.34 (s, 1F), −134.53 (d, J=9.6 Hz, 1F); MS 512 (M+1, 100%). CHN: Theoretical: C 53.06%, H 5.16%, N 7.42% (calc'd as 1.0 H₂O). Found: C 53.03%, H 4.82%, N 7.22, Cl 6.64%.

### [4-(5-Aminomethyl-2-fluorophenyl)piperidine-1-yl][7-fluoro-1-(2-methoxyethyl)-4-trifluoromethoxy-1H-indol-3-yl]methanone Benzoate (10 benzoate salt)

A 20-L glass-jacketed reactor already containing a toluene solution assumed to contain [4-(5-aminomethyl-2-fluorophenyl)piperidine-1-yl][7-fluoro-1-(2-methoxyethyl)-4-trifluoromethoxy-1H-indol-3-yl]methanone (1320 g, 2.58 mol) is stirred and heated to 61° C. Benzoic acid (316 g, 2.58 mol) is added and, after all the benzoic acid has dissolved, cyclohexane (6.04 L) is added. The reaction is heated to 77° C. where it is seeded with [4-(5-aminomethyl-2-fluorophenyl)piperidine-1-yl][7-fluoro-1-(2-methoxyethyl)-4-trifluoromethoxy-1H-indol-3-yl]methanone benzoate (0.100 g) from a preceding batch. The crystallization progresses at 77° C. and after 15 min, the reaction is cooled at a ramp of −10° C./h. When the reaction reaches 61° C., both the stirring and the cooling are stopped and the reaction is allowed to cool to rt. After standing overnight, stirring is resumed and the product is collected by filtration. The filter cake is washed with a solvent mixture prepared from toluene (3 L) and cyclohexane (1.5 L). After drying partially by suction, the product is transferred to a drying oven where it is dried at 40° C. affording [4-(5-aminomethyl-2-fluorophenyl)piperidine-1-yl][7-fluoro-1-(2-methoxyethyl)-4-trifluoromethoxy-1H-indol-3-yl]methanone benzoate as a colorless solid: 1408.8 g (86%), mp=156-159° C. Elemental analysis: Calculated for C₂₅H₂₆F₅N₃O₃.C₇H₆O₂: C, 60.66; H, 5.09; N, 6.63. Found: C, 60.44; H, 5.01; N, 6.87. Infrared spectral features (cm-1): 1612, 1526, 1511, 1501, 1394, 1362, 1256, 1232, 1211, 1158, 1117, 999, 826.

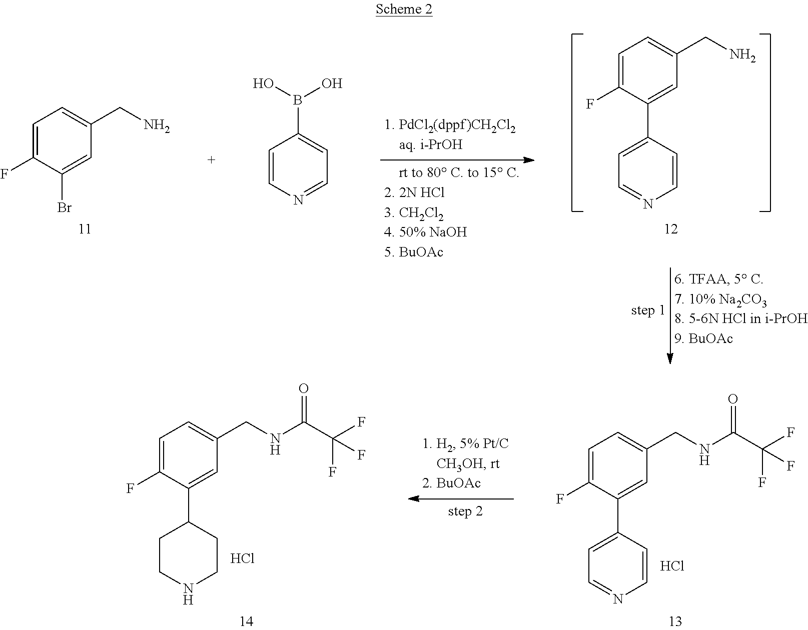

3-Bromo-4-fluorobenzylamine hydrochloride (Wychem) is reacted with pyridine-4-boronic acid (Clariant or Boron Molecular) in an alcoholic solvent with a boiling point of at least that of isopropyl alcohol, such as n-propyl alcohol, n-butyl alcohol and the like; polar aprotic solvent such as dimethylformamide, 1-methyl-2-pyrrolidone, dimethylsulfoxide, and the like etheral solvent such as 2-methyltetrahydrofuran, dimethoxyethane, and the like. Compound 12 and compound 13 in mixture of any of the above mentioned solvents and water in the presence of a suitable catalyst such as 1,1′-bis(diphenylphosphino)ferrocene-palladium(II)dichloride dichloromethane complex (PdCl₂dppf-CH₂Cl₂), Pd(PPh₃)₄, PdCl₂(PPh₃)₂, Pd(dtbpf)Cl₂, and the like with sufficient heating from about 70° C. to the temperature of the boiling point of the Suzuki coupling reaction mixture provides the pyridine.

This pyridine is converted to the trifluoroacetamide compound 2,2,2-trifluoro-N-(4-fluoro-3-pyridin-4-yl-benzyl)-acetamide hydrochloride under trifluoroacetylating conditions using a suitable trifluoroacetylating agent such as trifluoroacetic anhydride, trifluoroacetyl fluoride, pentafluorophenyl trifluoroacetate and the like, in a trifluoroacetylating solvent such as an ester solvent such as ethyl acetate, isopropyl acetate, or the like; an aromatic hydrocarbon solvent such as toluene, or the like; a chlorinated hydrocarbon solvent such as methylene chloride, 1,2-dichloroethane, or the like, at a trifluoroacetylation reaction temperature of about −20 to about 30° C., followed by treatment with hydrochloric acid.

2,2,2-Trifluoro-N-(4-fluoro-3-pyridin-4-yl-benzyl)-acetamide hydrochloride is reduced to under hydrogenation conditions to compound 14 by treatment with hydrogen in the presence of a hydrogenation catalyst means PtO₂, Pd/C, Pd(OH)₂, Rh/C and the like, with or without added inorganic acid such as HCl and the like, or organic acid such as acetic acid and the like, in a hydrogenation reaction solvent such as an alcohol solvent such as ethanol, isopropyl alcohol and the like; or acetic acid; or a mixture of an alcohol solvent or acetic acid and water, at hydrogenation reaction temperature of from about 10 to about 60° C., and hydrogenation pressure of from about 20 to about 1000 psi.

The compound of the present invention is basic, and such compound is useful in the form of the free base or in the form of a pharmaceutically acceptable acid addition salt thereof.

Acid addition salts may be a more convenient form for use; and in practice, use of the salt form inherently amounts to use of the free base form. The acids which can be used to prepare the acid addition salts include preferably those which produce, when combined with the free base, pharmaceutically acceptable salts, that is, salts whose anions are non-toxic to the patient in pharmaceutical doses of the salts, so that the beneficial inhibitory effects inherent in the free base are not vitiated by side effects ascribable to the anions. Although pharmaceutically acceptable salts of said basic compound is preferred, all acid addition salts are useful as sources of the free base form even if the particular salt, per se, is desired only as an intermediate product as, for example, when the salt is formed only for purposes of purification, and identification, or when it is used as intermediate in preparing a pharmaceutically acceptable salt by ion exchange procedures. Pharmaceutically acceptable salts within the scope of the invention include those derived from mineral acids and organic acids, and include hydrohalides, e.g. hydrochloride and hydrobromide, sulfates, phosphates, nitrates, sulfamates, acetates, citrates, lactates, tartrates, malonates, oxalates, salicylates, propionates, succinates, fumarates, maleates, methylene-bis-b-hydroxynaphthoates, benzoates, tosylates, gentisates, isethionates, di-p-toluoyltartrates, methanesulfonates, ethanesulfonates, benzenesulfonates, p-toluenesulfonates, cyclohexylsulfamates and quinates. A more particular salt is salt of the compound of formula I is the hydrochloride salt. Another particular salt of the present invention is the fumarate of the compound of formula I. A preferred pharmaceutically acceptable salt of the present invention is the benzoate of the compound of formula I.

As well as being useful in itself as an active compound, salts of the compound of the invention are useful for the purposes of purification of the compound, for example by exploitation of the solubility differences between the salts and the parent compound, side products and/or starting materials by techniques well known to those skilled in the art.

According to a further feature of the invention, the acid addition salt of the compound of this invention may be prepared by reaction of the free base with the appropriate acid, by the application or adaptation of known methods. For example, the acid addition salts of the compound of this invention may be prepared either by dissolving the free base in water or aqueous alcohol solution or other suitable solvents containing the appropriate acid and isolating the salt by evaporating the solution, or by reacting the free base and acid in an organic solvent, in which case the salt separates directly or can be obtained by concentration of the solution.

The acid addition salts of the compound of this invention can be regenerated from the salts by the application or adaptation of known methods. For example, the parent compound of the invention can be regenerated from their acid addition salts by treatment with an alkali, e.g. aqueous sodium bicarbonate solution or aqueous ammonia solution.

In particular, the monobenzoate of Compound A is preferred.

The starting materials and intermediates may be prepared by the application or adaptation of known methods, for example methods as described in the Reference Examples or their obvious chemical equivalents.

The present invention is also directed to some intermediates in the above scheme 1 and, as such, the processes described herein for their preparation constitute further features of the present invention.

### List of Abbreviations

As used above, and throughout the description of the invention, the following abbreviations, unless otherwise indicated, shall be understood to have the following meanings:
ACN acetonitrile
AIBN 2,2′-azobisisobutyronitrile
bid twice daily
BOC or Boc tent-butyl carbamate
BOP benzotriazol-1-yl-oxytris(dimethylamino)phosphonium
n-Bu₃ SnH tri-n-butyltin hydride
t-Bu tert-butyl
Cbz benzyl carbamate
PTC phase transfer catalyst
DAST (diethylamino) sulfur trifluoride (Et₂NSF₃)
DCC dicyclohexylcarbodiimide
DCM dichloromethane (CH₂Cl₂)
DIC 1,3-diisopropylcarbodiimide
DIPEA diisopropylethylamine
DMAP 4-(N,N-dimethylamino)pyridine
DMP reagent Dess-Martin Periodinane reagent
DMF dimethylformamide
DMSO dimethylsulfoxide
EA elemental analysis
EDCI 1-ethyl-3-(3-dimethylaminopropyl)carbodiimide HCl
eq equivalent(s)
Et ethyl
Et₂O diethyl ether
EtOH ethanol
EtOAc ethyl acetate
FMOC 9-fluorenylmethoxycarbonyl
HOAt 1-hydroxy-7-azabensotriazole
HOBT 1-hydroxybenztriazole
HOSu N-hydroxysuccinamide
HPLC high performance liquid chromatography
LAH lithium aluminum anhydride
Me methyl
MeI methyliodide
MeOH methanol
MeOC(O) methyl chloroformate
MOMCI methoxymethylchloride
MOM methoxymethyl
MS mass spectroscopy
NaBH₄ sodium borohydride
Na₂C₄H₄O₆ sodium tartrate
NMR nuclear magnetic resonance
P Polymer bond
PO per oral administration
PyBOP benzotriazole-1-yl-oxytris-pyrrolidino-phosphonium hexafluorophosphate
TBD 1,5,7-triazabicyclo[4.4.0]-dec-5-ene
RP-HPLC reverse phase-high pressure liquid chromatography
TBSCI tert-butyldimethylsilyl chloride
TCA trichloroacetic acid
TFA trifluoroacetic acid
Tf₂O triflate anhydride
THF tetrahydrofuran
THP tetrahydropyran
TLC thin layer chromatography

### Definitions

As used above, and throughout the description of the invention, the following terms, unless otherwise indicated, shall be understood to have the following meanings:

“Acid bioisostere” means a group which has chemical and physical similarities producing broadly similar biological properties to a carboxy group (see Lipinski, Annual Reports in Medicinal Chemistry, “Bioisosterism In Drug Design” 21, 283 (1986); Yun, Hwahak Sekye, “Application of Bioisosterism To New Drug Design” 33, 576-579, (1933); Zhao, Huaxue Tongbao, “Bioisosteric Replacement And Development Of Lead Compounds In Drug Design” 34-38, (1995); Graham, Theochem, “Theoretical Studies Applied To Drug Design ab initio Electronic Distributions In Bioisosteres” 343, 105-109, (1995)). Exemplary acid bioisosteres include —C(O)—NHOH, —C(O)—CH2OH, —C(O)—CH2SH, —C(O)—NH—CN, sulfo, phosphono, alkylsulfonylcarbamoyl, tetrazolyl, arylsulfonylcarbamoyl, N-methoxycarbamoyl, heteroarylsulfonylcarbamoyl, 3-hydroxy-3-cyclobutene-1,2-dione, 3,5-dioxo-1,2,4-oxadiazolidinyl or hydroxyheteroaryl such as 3-hydroxyisoxazolyl, 3-hydoxy-1-methylpyrazolyl and the like.

“Effective amount” is means an amount of a compound/composition according to the present invention effective in producing the desired therapeutic effect.

“Hydrate” means a solvate wherein the solvent molecule {s) is/are H₂O.

“Patient” includes both human and other mammals.

“Pharmaceutically acceptable ester” refers to esters that hydrolyze in vivo and include those that break down readily in the human body to leave the parent compound or a salt thereof, Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Exemplary esters include formates, acetates, propionates, butyrates, acrylates, ethylsuccinates, and the like.

“Pharmaceutically acceptable prodrugs” as used herein refers to those prodrugs of the compounds of the present invention which are, within the scope of sound medical judgment, suitable for use in contact with the tissues of patients with undue toxicity, irritation, allergic response, and the like, commensurate with a reasonable benefit/risk ratio, and effective for their intended use of the compounds of the invention. The term “prodrug” refers to compounds that are rapidly transformed in vivo to yield the parent compound of the above formula, for example by hydrolysis in blood. Functional groups that may be rapidly transformed, by metabolic cleavage, in vivo form a class of groups reactive with the carboxyl group of the compounds of this invention. They include, but are not limited to such groups as alkanoyl (such as acetyl, propanoyl, butanoyl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds of this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion is provided in Design of Prodrugs, H. Bundgaard, ed., Elsevier (1985); Methods in Enzymology; K. Widder et al, Ed., Academic Press, 42, 309-396 (1985); A Textbook of Drug Design and Development, Krogsgaard-Larsen and H. Bandaged, ed., Chapter 5; “Design and Applications of Prodrugs” 113-191 (1991); Advanced Drug Delivery Reviews, H. Bundgard, 8 , 1-38, (1992); J. Pharm. Sci., 77,285 (1988); Chem. Pharm. Bull., N. Nakeya et aI, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, which are incorporated herein by reference.

“Pharmaceutically acceptable salts” refers to the relatively non-toxic, inorganic and organic acid addition salts, and base addition salts, of compounds of the present invention. These: salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-B-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

“Solvate” means a physical association of a compound of this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, and the like.

“Treating” and “Treatment” mean administration of a compound to either ameliorate a disease condition or disorder, or prevent a disease condition or disorder. Or, the slowing of the progression of the disease condition or disorder. And these also refer to reducing susceptibility to a disease condition or disorder. The terms also include but are not limited to palliative therapy that is non-curative.

### Embodiments

With reference to inventions described herein, below are particular embodiments related thereto.

A particular embodiment of the invention is a method of treating a dermatological allergic condition, such as atopic dermatitis, comprising: administering to a patient in need thereof an effective amount of a compound of Formula I or a corresponding N-oxide, prodrug, pharmaceutically acceptable salt or solvate thereof.

Another particular embodiment of the invention is a pharmaceutical composition for treating a dermatological allergic condition, such as atopic dermatitis, comprising a compound of formula I, or a corresponding N-oxide, prodrug, pharmaceutically acceptable salt or salt thereof, in combination with a pharmaceutically acceptable excipient.

Yet another embodiment of the invention is a method of treating a dermatological allergic condition, such as atopic dermatitis, comprising: administering to a patient in need thereof an effective amount of a compound which is a beta-tryptase inhibitor.

The compounds of the invention optionally are supplied as salts. Those salts that are pharmaceutically acceptable are of particular interest since they are useful in administering the foregoing compounds for medical purposes. Salts that are not pharmaceutically acceptable are useful in manufacturing processes, for isolation and purification purposes, and in some instances, for use in separating stereoisomeric forms of the compounds of this invention. The latter is particularly true of amine salts prepared from optically active amines.

Where the compound of the invention contains a carboxy group, or a sufficiently acidic bioisostere, base addition salts may be formed and are simply a more convenient form of use; and in practice, use of the salt form inherently amounts to use of the free acid form.

Also, where the compound of the invention contains a basic group, or a sufficiently basic bioisostere, acid addition salts may be formed and are simply a more convenient form for use; and in practice, use of the salt form inherently amounts to use of the free base form.

Another object of the present invention is to provide a pharmaceutical composition comprising, a pharmaceutically effective amount of a compound of formula 1 and pharmaceutically acceptable carrier or diluent.

It is another object of the invention to provide a pharmaceutical composition which is effective, in and of itself, for utilization in a beneficial combination therapy because it includes a plurality of active ingredients which may be utilized in accordance with the invention.

The invention also provides kits or single packages combining two or more active ingredients useful in treating or preventing macular degeneration in a patient. A kit may provide (alone or in combination with a pharmaceutically acceptable diluent or carrier), the compound of formula 1 and the additional active ingredient (alone or in combination with diluent or carrier).

Compounds of formula I may be prepared by the application or adaptation of known methods as used heretofore or described in the literature, or by methods disclosed herein.

The amount of the compound of Formula I in any of the foregoing applications can be a pharmaceutically effective amount, a suboptimal effective amount, or combinations thereof, so long as the final combination of ingredients comprises a pharmaceutically effective amount of compounds that is effective in treating or preventing macular degeneration in a patient.

### Pharmacology

Compounds according to the invention as described herein as being useful for being able to inhibit beta-tryptase, and are also useful for treating inflammatory bowel disease.

A particular aspect of the invention provides for a compound according to the invention to be administered in the form of a pharmaceutical composition, though the compound may be administered alone. “Pharmaceutical composition” means a composition comprising a compound of formula 1 and at least one component selected from the group comprising pharmaceutically acceptable carriers, diluents, coatings, adjuvants, excipients, or vehicles, such as preserving agents, fillers, disintegrating agents, wetting agents, emulsifying agents, emulsion stabilizing agents, suspending agents, isotonic agents, sweetening agents, flavoring agents, perfuming agents, coloring agents, antibacterial agents, antifungal agents, other therapeutic agents, lubricating agents, adsorption delaying or promoting agents, and dispensing agents, depending on the nature of the mode of administration and dosage, forms. The compositions may be presented in the form of tablets, pills, granules, powders, aqueous solutions or suspensions, injectable solutions, elixirs or syrups. Exemplary suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Exemplary antibacterial and antifungal agents for the prevention of the action of microorganisms include parabens, chlorobutanol, phenol, sorbic acid, and the like. Exemplary isotonic agents include sugars, sodium chloride and the like. Exemplary adsorption delaying agents to prolong absorption include aluminum monostearate and gelatin. Exemplary adsorption promoting agents to enhance absorption include dimethyl sulfoxide and related analogs. Exemplary carriers, diluents, solvents, vehicles, solubilizing agents, emulsifiers and emulsion stabilizers, include water, chloroform, sucrose, ethanol, isopropyl alcohol, ethyl carbonate, ethyl acetate, benzyl alcohol, tetrahydrofurfuryl alcohol, benzyl benzoate, polyols, propylene glycol, 1,3-butylene glycol, glycerol, polyethylene glycols, dimethylformamide, Tween® 60, Span® 60, cetostearyl alcohol, myristyl alcohol, glyceryl mono-stearate and sodium lauryl sulfate, fatty acid esters of sorbitan, vegetable oils (such as cottonseed oil, groundnut oil, corn germ oil, olive oil, castor oil and sesame oil) and injectable organic esters such as ethyl oleate, and the like, or suitable mixtures of these substances. Exemplary excipients include lactose, milk sugar, sodium citrate, calcium carbonate, dicalcium phosphate. Exemplary disintegrating agents include starch, alginic acids and certain complex silicates. Exemplary lubricants include magnesium stearate, sodium lauryl sulfate, talc, as well as high molecular weight polyethylene glycols.

Other therapeutic agents may be used in combination with a compound of the present invention. Therapeutic agents used in combination with a compound of the present invention may be administered separately, simultaneously or sequentially. The choice of material in the pharmaceutical composition other than the compound of formula 1 is generally determined in accordance with the chemical properties of the active compound such as solubility, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulfate and talc may be used for preparing tablets.

The pharmaceutical compositions may be presented in assorted forms such as tablets, pills, granules, powders, aqueous solutions or suspensions, injectable solutions, elixirs or syrups.

“Liquid dosage form” means the dose of the active compound to be administered to the patient is in liquid form, for, example, pharmaceutically acceptable emulsions, solutions, suspensions, syrups and elixirs. In addition to the active compounds, the liquid dosage forms may contain inert diluents commonly used in the art, such solvents, solubilizing agents and emulsifiers.

Solid compositions may also be employed as fillers in soft and hard-filled gelatin capsules using such excipients as lactose or milk sugar as well as high molecular weight polyethylene glycols, and the like.

When aqueous suspensions are used they can contain emulsifying agents or agents which facilitate suspension.

The oily phase of the emulsion pharmaceutical composition may be constituted from known ingredients in a known manner. While the phase may comprise merely an emulsifier (otherwise known as an emulgent), it desirably comprises a mixture of at least one emulsifier with a fat or an oil or with both a fat and an oil. In a particular embodiment, a hydrophilic emulsifier is included together with a lipophilic emulsifier that acts as a stabilizer. Together, the emulsifier(s) with or without stabilizer(s) make up the emulsifying wax, and the way together with the oil and fat make up the emulsifying ointment base which forms the oily dispersed phase of the cream formulations.

If desired, the aqueous phase of the cream base may include, for example, a least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound that enhances absorption or penetration of the active ingredient through the skin or other affected areas.

The choice of suitable oils or fats for a formulation is based on achieving the desired properties. Thus the cream should preferably be a non-greasy, non-staining and washable product with suitable consistency to avoid leakage from tubes or other containers. Straight or branched chain, mono- or dibasic alkyl esters such as di-isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

In practice, a compound/pharmaceutical compositions of the present invention may be administered in a suitable formulation to humans and animals by topical or systemic administration, including oral, inhalational, rectal, nasal, buccal, sublingual, vaginal, colonic, parenteral (including subcutaneous, intramuscular, intravenous, intradermal, intrathecal and epidural), intracisternal and intraperitoneal. It will be appreciated that the preferred route may vary with for example the condition of the recipient.

“Pharmaceutically acceptable dosage forms” refers to dosage forms of the compound of the invention, and includes, for example, tablets, dragées, powders, elixirs, syrups, liquid preparations, including suspensions, sprays, inhalants tablets, lozenges, emulsions, solutions, granules, capsules and suppositories, as well as liquid preparations for injections, including liposome preparations. Techniques and formulations generally may be found in Remington's Pharmaceutical Sciences, Mack Publishing Co., Easton, Pa., latest edition.

“Formulations suitable for oral administration” may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tables may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compounds moistened with an inert liquid diluent. The tablets may optionally be coated or scored and may be formulated so as to provide slow or controlled release of the active ingredient therein.

Solid compositions for rectal administration include suppositories formulated in accordance with known methods and containing at least one compound of the invention.

If desired, and for more effective distribution, the compounds can be microencapsulated in, or attached to, a slow release or targeted delivery systems such as a biocompatible, biodegradable polymer matrices (e.g., poly(d,l-lactide co-glycolide)), liposomes, and microspheres and subcutaneously or intramuscularly injected by a technique called subcutaneous or intramuscular depot to provide continuous slow release of the compound(s) for a period of 2 weeks or longer. The compounds may be sterilized, for example, by filtration through a bacteria retaining filter, or by incorporating sterilizing agents in the form of sterile solid compositions which can be dissolved in sterile water, or some other sterile injectable medium immediately before use.

“Formulations suitable for nasal or inhalational administration” means formulations which are in a form suitable to be administered nasally or by inhalation to a patient. The formulation may contain a carrier, in a powder form, having a particle size for example in the range 1 to 500 microns (including particle sizes in a range between 20 and 500 microns in increments of 5 microns such as 30 microns, 35 microns, etc.). Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents. Inhalational therapy is readily administered by metered dose inhalers.

“Formulations suitable for oral administration” means formulations which are in a form suitable to be administered orally to a patient. The formulations may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

“Formulations suitable for parenteral administration” means formulations that are in a form suitable to be administered parenterally to a patient. The formulations are sterile and include emulsions, suspensions, aqueous and non-aqueous injection solutions, which may contain suspending agents and thickening agents and anti-oxidants, buffers, bacteriostats and solutes which render the formulation isotonic, and have a suitably adjusted pH, with the blood of the intended recipient.

“Formulations suitable for rectal or vaginal administrations” means formulations that are in a form suitable to be administered rectally or vaginally to a patient. Suppositories are a particular form for such formulations that can be prepared by mixing the compounds of this invention with suitable non-irritating excipients or carriers such as cocoa butter, polyethylene glycol or a suppository wax, which are solid at ordinary temperatures but liquid at body temperature and therefore, melt in the rectum or vaginal cavity and release the active component.

“Formulations suitable for systemic administration” means formulations that are in a form 20 suitable to be administered systemically to a patient. The formulation is preferably administered by injection, including transmuscular, intravenous, intraperitoneal, and subcutaneous. For injection, the compounds of the invention are formulated in liquid solutions, in particular in physiologically compatible buffers such as Hank's solution or Ringer's solution. In addition, the compounds may be formulated in solid form and redissolved or suspended immediately prior to use. Lyophilized forms are also included. Systematic administration also can be by transmucosal or transdermal means, or the compounds can be administered orally. For transmucosal or transdermal administration, penetrants appropriate to the barrier to be permeated are used in the formulation. Such penetrants are generally known in the art, and include, for example, bile salts and fusidic acid derivatives for transmucosal administration. In addition, detergents may be used to facilitate permeation. Transmucosal administration may be through use of nasal sprays, for example, or suppositories. For oral administration, the compounds are formulated into conventional oral administration forms such as capsules, tablets, and tonics.

“Formulations suitable for topical administration” means formulations that are in a form suitable to be administered topically to a patient. The formulation may be presented as a topical ointment, salves, powders, sprays and inhalants, gels (water or alcohol based), creams, as is generally known in the art, or incorporated into a matrix base for application in a patch, which would allow a controlled release of compound through the transdermal barrier. When formulated in an ointment, the active ingredients may be employed with either a paraffinic or a water-miscible ointment base. Alternatively, the active ingredients may be formulated in a cream with an oil-in-water cream base. Formulations suitable for topical administration in the eye include eye drops wherein the active ingredient is dissolved or suspended in a suitable carrier, especially an aqueous solvent for the active ingredient. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

“Solid dosage form” means the dosage form of the compound of the invention is solid form, for example capsules, tablets, pills, powders, dragées or granules. In such solid dosage forms, the compound of the invention is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alginates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, (i) lubricants, as for example, talc, calcium stearate, magnesium stearate, solid polyethylene glycols, sodium lauryl sulfate, (j) opacifying agents, (k) buffering agents, and agents which release the compound(s) of the invention ‘in a certain part of the intestinal tract in a delayed manner.

Actual dosage levels of active ingredient(s) in the compositions of the invention may be varied so as to obtain an amount of active ingredient(s) that is (are) effective to obtain a desired therapeutic response for a particular composition and method of administration for a patient. A selected dosage level for any particular patient therefore depends upon a variety of factors including the desired therapeutic effect, on the route of administration, on the desired duration of treatment, the etiology and severity of the disease, the patient's condition, weight, sex, diet and age, the type and potency of each active ingredient, rates of absorption, metabolism and/or excretion and other factors.

Total daily dose of the compounds of this invention administered to a patient in single or divided doses may be in amounts, for example, of from about 0.001 to about 100 mg/kg body weight daily and preferably 0.01 to 10 mg/kg/day. For example, in an adult, the doses are generally from about 0.01 to about 100, preferably about 0.01 to about 10, mg/kg body weight per day by inhalation, from about 0.01 to about 100, preferably 0.1 to 70, more especially 0.5 to 10, mg/kg body weight per day by oral administration, and from about 0.01 to about 50, preferably 0.01 to 10, mg/kg body weight per day by intravenous administration. The percentage of active ingredient in a composition may be varied, though it should constitute a proportion such that a suitable dosage shall be obtained. Dosage unit compositions may contain such amounts of such submultiples thereof as may be used to make up the daily dose. Obviously, several unit dosage forms may be administered at about the same time. A dosage may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. It goes without saying that, for other patients, it will be necessary to prescribe not more than one or two doses per day.

The formulations can be prepared in unit dosage form by any of the methods well known in the art of pharmacy. Such methods include the step of bringing into association the active ingredient with the carrier that constitutes one or more accessory ingredients. In general the formulations are prepared by uniformly and intimately bringing into association the active ingredient with liquid carriers or finely divided solid carriers or both, and then, if necessary, shaping the product.

The formulations may be presented in unit-dose or multi-dose containers, for example sealed ampoules and vials with elastomeric stoppers, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Compounds within the scope of the present invention exhibit marked pharmacological activities according to tests described in the literature and below, which tests results are believed to correlate to pharmacological activity in humans and other mammals.

The chemical reactions described in the references cited above are generally disclosed in terms of their broadest application to the preparation of the compounds of this invention.

Occasionally, the reactions may not be applicable as described to each compound included within the scope of compounds disclosed herein. The compounds for which this occurs will be readily recognized by those skilled in the art. In all such cases, either the reactions can be successfully performed by conventional modifications known to those skilled in the art, e.g., by appropriate protection of interfering groups, by changing to alternative conventional reagents, by routine modification of reaction conditions, and the like, or other reactions disclosed herein or otherwise conventional will be applicable to the preparation of the corresponding compounds of this invention. In all preparative methods, all starting materials are known or readily preparable from known starting materials.

The regimen for treating a patient suffering from a dermatological allergic condition, such as atopic dermatitis, with the compound and/or compositions of the present invention is selected in accordance with a variety of factors, including the age, weight, sex, diet, and medical condition of the patient, the severity of the condition, the route of administration, pharmacological considerations such as the activity, efficacy, pharmacokinetic, and toxicology profiles of the particular compounds employed, and whether a drug delivery system is utilized. Administration of the drug combinations disclosed herein should generally be continued over a period until acceptable, indicating that has been controlled or eradicated. Patients undergoing treatment with the drug combinations disclosed herein can be routinely monitored by conventional methods of measuring kidney function to determine the effectiveness of therapy. Continuous analysis of the data obtained by these methods permits modification of the treatment regimen during therapy so that optimal amounts of each component in the combination are administered, and so that the duration of treatment can be determined as well. Thus, the treatment regimen/dosing schedule can be rationally modified over the course of therapy so that the lowest amounts of each of the compounds used in combination which together exhibit satisfactory effectiveness are administered, and so that administration of such compounds in combination is continued only so long as is necessary to successfully treat a dermatological allergic condition, such as atopic dermatitis.

### EXPERIMENTAL EXAMPLE

### Animal Information

The species used for this study were male, nonhuman primates, Macaca fascicularis 5 to 10 years of age. Each animal was identified by a unique number tattoo located on its chest. The group size was 12. Animals were supplied by Charles River and housed under conditions outlined in the NIH Guide for Care and Use of Laboratory Animals in compliance with the USDA Laboratory Animal Welfare Act, in a fully accredited AAALAC facility. Purina (product 5038) primate chow was fed twice daily, supplemented with fruit, vegetables and treats. Water was available ad libitum.

The animal use protocol that was followed for this study was approved by the Institutional Animal Care and Use Committee.

### Compound Information

Compound A initially referred to as the salt form (hydrochloride salt), was synthesized by the scheme described above. Salt/Active Substance Ratio (w/w): 1.07.

Diphenhydramine hydrochloride (Bioniche Pharma, catalog number 1084899, lot number 070709) was used as the positive control.

### Formulations

Compound A formulated in a solution containing 0.22 mg/ml A sum (Ascaris sum) in phosphate buffered saline was dosed at 0.004, 0.0004, 0.00004, and 0.000004 mg per injection site at a volume of 40 μl.

Diphenhydramine hydrochloride was formulated in a solution containing 0.22 mg/ml A suum in phosphate buffered saline. Diphenhydramine hydrochloride was dosed at 0.4 mg per injection site at a volume of 40 μl.

### Antigen Formulation

Ascaris suum extract (part number XPB33X1A5, lot 30575) was supplied by Greer Laboratories. The extract was reconstituted with distilled water to a final concentration of 10 mg/ml of stock solution.

### Procedure

Animals were anesthetized by an intramuscular (IM) injection of 5 to 10 mg/kg ketamine mixed with 0.5 to 0.75 mg/kg medetomidine. The animal's chest and abdomen were shaved and wiped clean with alcohol. The following injections (40 μl each) were given intradermally:
PBS (phosphate buffered saline)
Ascaris 8.8×10⁻³ mg/ml
Ascaris 8.8×10⁻³ mg/ml+0.4 mg Diphenhydramine hydrochloride
Ascaris 8.8×10⁻³ mg/ml+0.004 mg SAR160719
Ascaris 8.8×10⁻³ mg/ml+0.0004 mg SAR160719
Ascaris 8.8×10⁻³ mg/ml+0.00004 mg SAR160719
Ascaris 8.8×10⁻³ mg/ml+0.000004 mg SAR160719

The reactions in the form of a wheal were measured 15 minutes after injection using vernier calipers. The wheal was measured along two diameters (D1 and D2) perpendicular to one another. The area of the wheal was calculated using the formula:

Area(mm²)=((D1+D2)/4)²×3.142

Following the measurement, hydrocortisone cream was applied to all injection sites. Diphenhydramine hydrochloride 2 mg/kg was administered (IM) as well as Atropine 0.1 mg/kg subcutaneously. The animals were reversed with atipamezole hydrochloride (IM), recovered in their home cages and monitored for any further allergic reactions or itching at the injection sites.

### Statistical Analysis

Mixed model with repeated measures was used to analyze the data, with wheal size for different treatment groups as the repeated measures, treatment as fixed effects, and animal as random effect. The correlation structure was selected to be compound symmetry. Robust covariance estimate was used to obtain the results.

### Results

Compound A co-injected with A suum intradermally reduced antigen-induced wheal formation when measured 15 minutes after injection. The mean wheal area (±SE) of the A suum injection site was 119.626±10.175 mm². Compound A significantly reduced mean wheal area at the 0.0004 mg (97.680±8.012 mm²; p=0.0475) and 0.004 mg (102.390±9.645 mm²; p=0.0171) doses. Diphenhydramine hydrochloride co-injected with A suum significantly reduced mean wheal area at 0.4 mg (84.196±7.364 mm²; p=0.0005).

The results are displayed in Table I below.

TABLE 1
Individual calculated wheal area (mm²)

| | | | A suum 8.8 × 10⁻³ + | | | | |
|---|---|---|---|---|---|---|---|
| | | | Diphenhydramine | | | | |
| | | A suum | hydrochloride | Compound A | Compound A | Compound A | Compound A |
| Animal ID | PBS | 8.8 × 10⁻³ mg | 0.4 mg | 0.004 mg | 0.0004 mg | 0.00004 mg | 0.000004 mg |
| 14-230 | 22.065 | 93.325 | 87.428 | 76.211 | 56.752 | 134.800 | 115.005 |
| 14-228 | 16.621 | 43.014 | 94.183 | 84.960 | 95.046 | 106.610 | 89.932 |
| 91-165 | 35.261 | 103.882 | 76.211 | 67.938 | 112.171 | 67.209 | 110.302 |
| 63-103 | 27.809 | 133.773 | 116.914 | 115.005 | 110.302 | 86.601 | 84.145 |
| 113-215 | 0.000 | 166.292 | 147.430 | 159.506 | 158.388 | 168.586 | 132.750 |
| 14-233 | 18.098 | 110.302 | 62.219 | 91.621 | 101.190 | 90.774 | 121.754 |
| 54-1288 | 0.000 | 115.957 | 73.148 | 84.960 | 103.882 | 106.610 | 86.601 |
| 54-1293 | 52.175 | 142.099 | 82.527 | 131.730 | 107.527 | 81.723 | 104.788 |
| 14-222 | 34.216 | 97.655 | 58.773 | 88.259 | 87.428 | 90.774 | 134.800 |
| 54-1279 | 24.195 | 130.715 | 67.209 | 164.014 | 90.774 | 107.527 | 133.773 |
| 63-113 | 38.490 | 121.754 | 78.550 | 99.415 | 100.300 | 93.325 | 85.779 |
| 103-350 | 0.000 | 176.738 | 65.764 | 65.047 | 48.404 | 79.337 | 66.485 |
| Mean | 22.413 | 119.626 | 84.196 | 102.390 | 97.680 | 101.157 | 105.510 |
| SE | 4.800 | 10.175 | 7.364 | 9.645 | 8.012 | 7.917 | 6.597 |
| p Value | <0.00001 | N/A | 0.0005 | 0.0171 | 0.0475 | 0.0899 | 0.2578 |

N/A = Not Applicable

The present invention may be embodied in other specific forms without departing from the spirit or essential attributes thereof.

What is claimed is:

1. A method of treating a dermatological allergic condition, comprising: administering to a patient in need thereof an effective amount of a compound of Formula I: 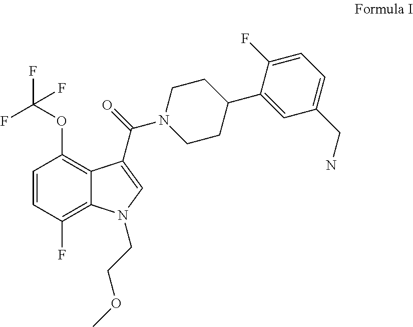
or a pharmaceutically acceptable salt thereof.

2. A pharmaceutical composition for treating a dermatological allergic condition, comprising a compound of formula I as defined in claim 1, or a pharmaceutically acceptable salt thereof, in combination with a pharmaceutically acceptable excipient.

3. The method according to claim 1, wherein the condition is an atopic skin disease, selected from atopic dermatitis, eczema, psoriasis, chronic urticaria and alopecia.

4. The method according to claim 3, wherein the compound is administered as the monobenzoate salt.
